(19) United States
(12) Patent Application Publication
(10) Pub. No.: US 2012/0035099 A1
(43) Pub. Date: Feb. 9, 2012

(54) FGF21 ANALOGUES AND DERIVATIVES

(75) Inventors: WILLIAM PATRICK GARIBAY, HOLTE (DK); HELLE WÖLDIKE, LYNGE (DK); XUJIA ZHANG, BEIJING (CN); HENNING THØGERSEN, FARUM (DK); PETER KRESTEN NIELSEN, HOLTE (DK); BIRGITTE ANDERSEN, MALOV (DK); JISHU WANG, BEIJING (CN); KRISTIAN SASS BAK-JENSEN, KOBENHAVN V (DK); TINA MØLLER TAGMOSE, BALLERUP (DK)

(73) Assignee: NOVO NORDISK A/S, BAGSVAERD (DK)

(21) Appl. No.: 13/154,081

(22) Filed: Jun. 6, 2011

Related U.S. Application Data
(60) Provisional application No. 61/373,290, filed on Aug. 13, 2010.

(30) Foreign Application Priority Data
Jun. 11, 2009 (EP) 09162521.0
Jun. 8, 2010 (EP) PCT/EP2010/057986
Jul. 21, 2010 (CN) PCT/CN2010/001099

(51) Int. Cl. A61K 38/18; A61P 3/10; A61P 1/16; A61P 3/04; A61P 9/00; A61P 3/00; C07K 14/50; A61P 3/06

(52) U.S. Cl. 514/4.8; 530/399; 514/6.9; 514/7.4; 514/9.1

(57) ABSTRACT

Derivatives of Fibroblast Growth Factor 21 which have improved properties for treating diabetes can be prepared by a recombinant process.

### CROSS-REFERENCE TO RELATED APPLICATIONS

This application claims priority to International Patent Application PCT/CN2010/001099, filed Jul. 21, 2010, and International Patent Application PCT/EP2010/057986, filed Jun. 8, 2010, which claimed priority of European Patent Application 09162521.0, filed Jun. 11, 2009 and U.S. Provisional Application 61/186,471, filed Jun. 12, 2009; this application further claims priority under 35 U.S.C. §119 of U.S. Provisional Application 61/373,290, filed Aug. 13, 2010. Each of these priority documents is incorporated by reference herein in its entirety.

### FIELD OF THIS INVENTION

The present invention relates to novel analogues of Fibroblast Growth Factor 21 (FGF21) and to derivatives thereof having a modifying moiety covalently attached. The invention also relates to pharmaceutical use of these analogues and derivatives, in particular for the treatment of diabetes, dyslipidemia, obesity, cardiovascular diseases, metabolic syndrome, and/or Non Alcoholic Fatty Liver Disease (NAFLD).

The derivatives of the invention are protracted, e.g. capable of maintaining a low blood glucose level for a longer period of time, capable of increasing the in vivo half-life of FGF21, and/or result in a lower clearance of FGF21.

### BACKGROUND OF THIS INVENTION

Fibroblast growth factors are polypeptides expressed in developing and adult tissues. They are involved in several physiological mechanisms including for example metabolic regulation and cellular differentiation. A whole family of more than twenty fibroblast growth factors exists (the FGF family). Three members of the FGF family including FGF19, FGF21, and FGF23 form a subfamily functioning as endocrine factors involved in metabolic regulation.

Fibroblast Growth Factor 21 or FGF-21, herein for short FGF21, is expressed preferentially in the liver and has been shown to exert hormone-like metabolic effects. For example, FGF21 has been demonstrated to activate glucose uptake in mouse adipocytes, to protect mice from diet induced obesity when over-expressed in transgenic mice, and to lower blood glucose and triglyceride levels when administered to diabetic rodents (Kharitonenkov et al., J. Clin. Invest. (2005), 115:1627-1635). The lowering effect of FGF21 on blood glucose and triglycerides has also been shown in diabetic monkeys. FGF21 was also able to decrease LDL and to increase HDL significantly in diabetic monkeys (Kharitonenkov et al., Endocrinology (2007), 148(2):774-81).

In diet induced obese mice and ob/ob mice, FGF21 was furthermore shown to lower body weight, predominantly by an increase in energy expenditure and a reduction in adiposity (Coskun et al., Endocrinology (2008), 149(12): 6018-6027).

Based on these results FGF21 has been suggested as a pharmacological agent with the potential to treat diabetes, dyslipidemia, obesity, cardiovascular diseases, and metabolic syndrome. Metabolic syndrome includes aspects like insulin resistance, dyslipidemia, visceral obesity and hypertension, see e.g. the definition of metabolic syndrome in Grundy et al., Circulation (2004), (109): 433-438.

FGF21 may furthermore be used as a pharmacological agent with a potential to treat Non Alcoholic Fatty Liver Disease (NAFLD), see Coskun et al. Endocrinology, 2008 cited above, and Xu et al., Diabetes (2009, 58(1):250-9, published electronically 7, Oct. 2008 ahead of print). NAFLD has been defined by Erickson, J. Lipid Res. (2008), published electronically 12, Dec. 2008 ahead of print. Yie et al. studied the role of the N- and C-termini of FGF21 in receptor interaction and activation, see FEBS Letters, 583 (2009), 19-24.

### OBJECTS OF THIS INVENTION

The object of this invention is to overcome or ameliorate at least one of the disadvantages of the prior art, or to provide a useful alternative.

Another aspect of this invention relates to the furnishing of analogues or derivatives of FGF21 which have improved properties for the treatment of diabetes, for example compared with human FGF21.

Another aspect of this invention relates to the furnishing of analogues or derivatives of FGF21 which have improved properties for the treatment of obesity, for example compared with human FGF21.

Another aspect of this invention relates to the furnishing of analogues or derivatives of FGF21 which have improved properties for the treatment of non-alcoholic fatty liver disease (NAFLD), for example compared with human FGF21.

Another aspect of this invention relates to the furnishing of analogues or derivatives of FGF21 which can relatively easy be prepared recombinant in E. coli.

Another aspect of this invention relates to the furnishing of analogues or derivatives of FGF21 being protected against N-terminal degradation.

Another aspect of this invention relates to the furnishing of analogues or derivatives of FGF21 which have increased potency with respect to glucose uptake in 3T3-L1 cells, for example compared with human FGF21.

Another aspect of this invention relates to the furnishing of analogues and derivatives of FGF-21 having increased mean half-life time compared with the mean half life time of Met-FGF-21, vide the test in example 9, below.

Further objects of this invention are to furnish compounds which can effectively be used to treat hypertension, critical illness, the metabolic syndrome, epilepsy, cancer, acromegaly, dyslipidemia (high TG, high LDL and low HDC) and cardiovascular diseases, e.g., atherosclerosis and hypercholesterolemia.

### DEFINITIONS

The sequence of the native human FGF21 protein is available from the UNIPROT database with α-cession no. Q9NSA1. The 209 amino acid precursor protein includes a signal peptide (amino acids 1-28) and a mature protein (amino acids 29-209). The mature protein is included herein as SEQ ID NO:1 (amino acids 1-181), and the signal peptide as SEQ ID NO:2 (amino acids 1-28).

An isoform or allelic form of native human FGF21 having a Pro instead of Leu in the mature protein at position 146 of SEQ ID NO:1 herein is known from, i.a., US2001012628 A1 (residue no. 174 of SEQ ID NO:2 in the published US application).

Another isoform having a shorter signal peptide in which Leu at position 23 of SEQ ID NO:2 herein is missing is known from WO 2003/011213 (see SEQ ID NO: 2 of the WO publication having a signal peptide of 27 amino acid residues).

Thus, particular examples of native human FGF21 are: SEQ ID NO:1, SEQ ID NO:1 having the substitution L146P, as well as any of these sequences preceded by the 27 or 28 amino acids signal peptide referred to above. Preferred examples of native human FGF21 are the mature parts, viz. SEQ ID NO:1 and the L146β isoform thereof.

The term “analogue” as referred to herein in the context of FGF21, i.e., an FGF21 analogue, refers to polypeptides that are or can be, deduced or derived from native FGF21, from SEQ ID NO:1 in particular, by modification of the amino acid sequence thereof. Such modification, amendment or change may include substitution, deletion, and/or addition of one or more amino acids. For example, an amino acid may be added at the N-terminus end.

The term “amino acid” or “amino acid residue” as referred to herein in the context of FGF21 modifications includes the twenty standard alpha-amino acids being used by cells in protein biosynthesis and specified by the genetic code, viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine (the amino acid residues being the corresponding residues from which hydrogen has been removed from an amino group and/or a hydroxy group has been removed from a carboxy group and hydrogen may have been removed from any mercapto group). Herein, an amino acid is preferably one which can be prepared by genetic engineering.

For the present purposes, the two recognized codes of the standard amino acids (one-letter and three-letter) are used interchangeably, or now and then the amino acid name is fully spelled out. These terms are of course considered fully equivalent (e.g., S=Ser=serine).

The term “derivative” as used herein refers to an analogue of FGF21 which has been covalently modified. The term is not limiting as such, rather descriptive, as it is intended to mark a distinction between changes made to the constituent FGF21 polypeptide as such (“analogues”), and the covalent binding of a side chain to the FGF21 compound, whereby the compound is “derivatised”. If desired, this term can be substituted with other general chemical terms, for example compound.

Nomenclature: Analogues and derivatives are named herein using, interchangeably, polypeptide nomenclature, organic chemical nomenclature, and chemical formulas, or mixtures thereof, whatever is deemed best suited for easing the understanding of the technical matter in question. For example, the derivative name S-71-({2-[2-(2-{2-[2-(2-{2-[(S)-4-carboxy-4-(17-carboxyheptadecanoylamino)butyrylamino]ethoxy}ethoxy)acetylamino]ethoxy}ethoxy)acetylamino]ethylcarbamoyl}methyl) [71C, 121Q, 166F, 168L, 173A, 174V, 179F] Ala-FGF21 means that [71C, 121Q, 166F, 168L, 173A, 174V, 179F] Ala-FGF21 is modified by {2-[2-(2-{2-[2-(2-{2-[(S)-4-carboxy-4-(17-carboxyheptadecanoylamino)butyrylamino]ethoxy}ethoxy)acetylamino]ethoxy}ethoxy)acetylamino]ethylcarbamoyl}methyl at the thiol group in Cys in position 71.

For example, a substitution in an analogue may be indicated as: “Original amino acid-position-substituted amino acid” (or as “position-substituted amino acid”). The three or one letter code may be used. Accordingly, the notation “S71C” or “Ser71Cys” means, that the analogue comprises a substitution of serine with cysteine in the amino acid position corresponding to the amino acid at position 71 in human FGF21 (SEQ ID NO:1).

Multiple modifications such as e.g. substitutions may be separated by commas (with a space after the comma), and if desired surrounded by brackets in order to make it clear that they belong to the same variant. Hence, the analogue designated [−1A, L166F, M168L, G174V, Y179F] FGF21 is human FGF21 having Ala (A) in position −1, Phe (F) in position 166, Leu (L) in position 168, Val (V) in position 174, and Phe (F) in position 179.

An extension can be described by reference to SEQ ID NO:1 by addition of position numbers (continued positive numbers in the C-terminal end and negative numbers in the N-terminal end) or, more simply, by adding the amino acids of the extension in question, using the correct sequence thereof, to the compound in question, which is then often given a trivial name, such as FGF21, again in order to ease the understanding of the relevant technical point. Hence, [−1A, L166F, M168L, G174V, Y179F] FGF21 can also be designated [L166F, M168L, G174V, Y179F] Ala-FGF21.

The term “compound” collectively covers analogues and derivatives.

### SUMMARY OF THE INVENTION

Briefly, this invention is as defined in the claims and clauses below.

The present invention relates to novel analogues and derivatives of FGF21. In said derivatives, a modifying group is covalently attached to the FGF21 analogue. The invention also relates to the use of said analogues and derivatives in pharmaceutical compositions, in particular for the treatment of diabetes, dyslipidemia, obesity, cardiovascular diseases, metabolic syndrome, and/or Non Alcoholic Fatty Liver Disease (NAFLD).

The derivatives of the invention are protracted, e.g. capable of maintaining a low blood glucose level for a longer period of time, capable of increasing the in vivo half-life of FGF21, and/or result in a lower clearance of FGF21. The derivatives of FGF21 retain satisfactory biological activity and may be administered less frequently than the parent FGF21 analogues. Furthermore, said derivatives have a reduced risk of deamidation.

### DETAILED DESCRIPTION OF THIS INVENTION

In one aspect, this invention relates to analogues of FGF21.

In one aspect, the analogues and derivatives of this invention are [−1A, L166F, M168L, G174V, Y179F] FGF21, analogues of [−1A, L166F, M168L, G174V, Y179F] FGF21 optionally containing one or more of the following amino acid substitutions (exchanges): 71C, 121Q, 173A and/or des181, optionally, having up to four further mutations and/or, optionally, the 179 and/or 180 amino acid is not present and derivatives of such analogues containing Cys in position 71 which derivatives have a group of the general formula HOOC—(CH₂)ₙ—CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71.

Hence, the above aspect covers, e.g., 1) analogues of [−1A, L166F, M168L, G174V, Y179F] FGF21 containing one or more of the following amino acid substitutions (exchanges): 71C, 121Q, 173A and/or des181, 2) analogues of [−1A, L166F, M168L, G174V, Y179F] FGF21 having up to four further mutations (apart from any mutation(s) selected from the group consisting of 71C, 121Q, 173A and/or des181), 3) analogues of [−1A, L166F, M168L, G174V, Y179F] FGF21 wherein the 179 and/or 180 amino acid is not present and 4) any combination of 1), 2) and/or 3).

The above expression “having up to four further mutations” means that up to four amino acid residues have been exchanged, inserted or cancelled in FGF-21, apart from any mutation(s) selected from the group consisting of 71C, 121Q, 173A and/or des181. Examples of such exchanges are the insertion of Pro in position 146.

FGF-21 analogues wherein the 179 and/or 180 amino acid is not present can also be designated des179 and/or des 180 analogues.

In another aspect, the analogues and derivatives of this invention are [−1A, 71C, L166F, M168L, G174V, Y179F] FGF21, analogues of [−1A, 71C, L166F, M168L, G174V, Y179F] FGF21 optionally containing one or more of the following amino acid substitutions (exchanges): 121Q, 173A and/or des181, optionally, having up to four further mutations and/or, optionally, the 179 and/or 180 amino acid is not present and derivatives of such analogues which derivatives have a group of the general formula HOOC—(CH₂), —CONH—CH(COOH)—CH₂—CH₂—CONH—(CH₂CH₂O)ₘ—CH₂—CONH—(CH₂—CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71.

In another aspect, the analogues of this invention are [−1A, L166F, S167G, M168L, G174V, Y179F, A180E] FGF21 and analogues of [−1A, L166F, S167G, M168L, G174V, Y179F, A180E] FGF21 additionally containing one or more of the following amino acid substitutions (exchanges): 71C, 121Q, 171L, 172E, 173A and/or des181. The meaning of the expression “analogues of [−1A, L166F, S167G, M168L, G174V, Y179F, A180E] FGF21 additionally containing one or more of the following amino acid substitutions (exchanges): 71C, 121Q, 171L, 172E, 173A and/or des181” is that, compared with human FGF21 (SEQ ID NO:1), said analogues contain Ala (A) in position −1, Phe (F) in position 166, Gly (G) in position 167, Leu (L) in position 168, Val (V) in position 174, Phe (F) in position 179, and Glu (E) in position 180 and, furthermore, either Cys (C) in position 71, Gln (O) in position 121, Leu (L) in position 171, Glu (E) in position 172, Ala (A) in position 173, and/or no amino acid residue in position 181.

In another aspect, the analogues of this invention are [−1A, S71C, L166F, S167G, M168L, G174V, Y179F, A180E] FGF21 and [−1A, S71C, L166F, S167G, M168L, G174V, Y179F, A180E]FGF21 analogues thereof additionally, contains one or more of the following amino acid substitutions (exchanges): 121Q, 171L, 172E, 173A and/or des181. Hence, these FGF21 analogues are [−1A, S71C, L166F, S167G, M168L, G174V, Y179F, A180E] FGF21 and FGF21 analogues thereof which in addition to Ala (A) in position −1, Cys (C) in position 71, Phe (F) in position 166, Gly (G) in position 167, Leu (L) in position 168, Val (V) in position 174, Phe (F) in position 179, and Glu (E) in position 180, additionally, contains either Gln (O) in position 121, Leu (L) in position 171, Glu (E) in position 172, Ala (A) in position 173, and/or no amino acid residue in position 181. Hence, the term “either Gln (O) in position 121, Leu (L) in position 171, Glu (E) in position 172, Ala (A) in position 173, and/or no amino acid residue in position 181” is herein also expressed as “121Q, 171L, 172E, 173A and/or des181”.

Hence, one analogue according to this invention is [−1A, S71C, L166F, S167G, M168L, G174V, Y179F, A180E] FGF21 which can, alternatively, be designated [S71C, L166F, S167G, M168L, G174V, Y179F, A180E] Ala-FGF21 or [71C, 166F, 167G, 168L, 174V, 179F, 180E] Ala-FGF21.

In another aspect, this invention relates to derivatives of FGF21 analogues. In one aspect, the derivatives of this invention are [−1A, S71C, L166F, S167G, M168L, G174V, Y179F, A180E] FGF21 and [−1A, S71C, L166F, S167G, M168L, G174V, Y179F, A180E] FGF21 analogues which FGF21 analogues additionally contain one or more of the following amino acid substitutions (exchanges): 121Q, 171L, 172E, 173A and/or des181, carrying a group of the general formula HOOC—(CH₂)ₙ—CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂-(modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71 of said FGF21 analogue. Herein, the group of the general formula HOOC—(CH₂), —CONH—CH(COOH)—CH₂—CH₂—CONN—(CH₂CH₂O)ₘ—CH₂—CONH—(CH₂CH₂O)ₚ—CH₂—CONH—(CH₂)_{q}—NHCO—CH₂—, wherein n, m, p and q are as defined herein, is designated a modifying moiety. It has been found that this modifying moiety has the capability of increasing the in vivo circulation time.

Compared with the analogues of FGF21, the derivatives of the above analogues of FGF21 have prolonged action.

A pharmaceutical composition comprising an analogue or a derivative of FGF21 may further comprise a pharmaceutically acceptable carrier. For injection, the carrier may be water, if desired supplemented with other materials, e.g. saline, such as physiological saline. Other pharmaceutically acceptable agents such as diluents and appropriate buffers may also be used. If desired, additional pharmaceutically acceptable agents such as emulsifiers, suspending agents, solvents, fillers, bulking agents, adjuvants, preservatives, antioxidants, colouring agents, and/or flavouring agents may also be used. The analogue or derivative of FGF21 may be used in the form of a purified polypeptide or a derivative thereof, or formulated using appropriate pharmaceutically acceptable excipients, as is known in the art. The pharmaceutical composition may be administered in any way as is known in the art, e.g. injected, for example intravenously (i.v.) or subcutaneously (s.c.).

The analogue or derivative of FGF21 may be included in the pharmaceutical composition in a therapeutically or prophylactically effective amount. The amount to be administered to the patient depends upon the therapeutic or prophylactic objective, such as the indication in question, the condition of the patient in need of treatment, the desired route of administration, etc. The skilled medical practitioner may have to adjust dosage and modify the administration depending on these factors, as is routine in the art. For example, the compounds of this invention can be administered once daily or one or more times per week.

### PREFERRED FEATURES OF THIS INVENTION

To sum up and supplement the above statements, the features and clauses of this invention are as follows:

1. [−1A, L166F, M168L, G174V, Y179F] FGF21, analogues of [−1A, L166F, M168L, G174V, Y179F] FGF21 optionally containing one or more of the following amino acid substitutions (exchanges): 71C, 121Q, 173A and/or des181, optionally, having up to four further mutations and/or, optionally, the 179 and/or 180 amino acid is not present and derivatives of such analogues containing Cys in position 71 which derivatives have a group of the general formula HOOC—(CH₂), —CONH—CH(COOH)—CH₂—CH₂-CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71.
2. [−1A, L166F, M168L, G174V, Y179F] FGF21, analogues of [−1A, L166F, M168L, G174V, Y179F] FGF21 optionally containing one or more of the following amino acid substitutions (exchanges): 71C, 121Q, 173A and/or des181, optionally, having up to four further mutations and/or, optionally, the 179 and/or 180 amino acid is not present, according to the previous clause.

3. [−1A, 71C, L166F, M168L, G174V, Y179F] FGF21, analogues of [−1A, 71C, L166F, M168L, G174V, Y179F] FGF21 optionally containing one or more of the following amino acid substitutions (exchanges): 121Q, 173A and/or des181, optionally, having up to four further mutations and/or, optionally, the 179 and/or 180 amino acid is not present and derivatives of such analogues which derivatives have a group of the general formula HOOC—(CH₂)ₙ—CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71, according to any one of the previous clauses.

4. [−1A, 71C, L166F, M168L, G174V, Y179F] FGF21, analogues of [−1A, 71C, L166F, M168L, G174V, Y179F] FGF21 optionally containing one or more of the following amino acid substitutions (exchanges): 121Q, 173A and/or des181, optionally, having up to four further mutations and/or, optionally, the 179 and/or 180 amino acid is not present, according to any one of the previous clauses to the extent possible.
5. [−1A, L166F, S167G, M168L, G174V, Y179F, A180E] FGF21, analogues of [−1A, L166F, S167G, M168L, G174V, Y179F, A180E] FGF21 additionally containing one or more of the following amino acid substitutions (exchanges): 71C, 121Q, 171L, 172E, 173A and/or des181 and derivatives of such analogues containing Cys in position 71 which derivatives have a group of the general formula HOOC—(CH₂)ₙ—CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71, according to any one of the previous clauses to the extent possible.
6. [−1A, L166F, S167G, M168L, G174V, Y179F, A180E] FGF21 and analogues of [−1A, L166F, S167G, M168L, G174V, Y179F, A180E] FGF21 additionally containing one or more of the following amino acid substitutions (exchanges): 71C, 121Q, 171L, 172E, 173A and/or des181.
7. [−1A, L166F, S167G, M168L, G174V, Y179F, A180E] FGF21, analogues of [−1A, L166F, S167G, M168L, G174V, Y179F, A180E] FGF21 additionally containing one or more of the following amino acid substitutions (exchanges): 71C, 121Q, 171L, 172E, 173A and/or des181 and/or, optionally, the 179 and/or 180 amino acid is not present and derivatives of such analogues containing Cys in position 71 which derivatives have a group of the general formula HOOC—(CH₂)ₙ—CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71, according to any one of the previous clauses to the extent possible.
8. [−1A, L166F, S167G, M168L, G174V, Y179F, A180E] FGF21, analogues of [−1A, L166F, S167G, M168L, G174V, Y179F, A180E] FGF21 additionally containing one or more of the following amino acid substitutions (exchanges): 71C, 121Q, 171L, 172E, 173A and/or des181 and/or, optionally, the 179 and/or 180 amino acid is not present.
9. [−1A, S71C, L166F, S167G, M168L, G174V, Y179F, A180E] FGF21 or analogues of [−1A, S71C, L166F, S167G, M168L, G174V, Y179F, A180E] FGF21 additionally containing one or more of the following amino acid substitutions (exchanges) 121Q, 171L, 172E, 173A and/or des181 and derivatives thereof having a group of the general formula HOOC—(CH₂), —CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71, according to any one of the previous clauses to the extent possible.
10. [−1A, S71C, L166F, S167G, M168L, G174V, Y179F, A180E] FGF21 or analogues of [−1A, S71C, L166F, S167G, M168L, G174V, Y179F, A180E] FGF21 additionally containing one or more of the following amino acid substitutions (exchanges) 121Q, 171L, 172E, 173A and/or des181, according to any one of the previous clauses to the extent possible.
11. The analogue according to any one of the previous clauses to the extent possible, containing only one of the following amino acid exchanges: Gln (O) in position 121, Leu (L) in position 171, Glu (E) in position 172, Ala (A) in position 173, or no amino acid in position 181.
12. The analogue according to any one of the previous clauses to the extent possible, containing only two of the following amino acid exchanges: Gln (O) in position 121, Leu (L) in position 171, Glu (E) in position 172, Ala (A) in position 173, or no amino acid in position 181.
13. The analogue according to any one of the previous clauses, to the extent possible, containing only three of the following amino acid exchanges: Gln (O) in position 121, Leu (L) in position 171, Glu (E) in position 172, Ala (A) in position 173, or no amino acid in position 181.
14. The analogue according to any one of the previous clauses to the extent possible, containing only four of the following amino acid exchanges: Gln (O) in position 121, Leu (L) in position 171, Glu (E) in position 172, Ala (A) in position 173, or no amino acid in position 181.
15. The analogue according to any one of the preceding clauses to the extent possible which is [−1A, S71C, L166F, S167G, M168L, G174V, Y179F, A180E] FGF21.
16. The analogue according to any one of the preceding clauses to the extent possible, which is [−1A, 71C, 121Q, 166F, 167G, 168L, 171L, 172E, 173A, 174V, 179F, 180E, des181] FGF21.
17. The analogue according to any one of the preceding clauses to the extent possible, which is [−1A, S71C, L166F, S167G, M168L, G174V, Y179F, A180E, des181] FGF-21.
18. The analogue according to any one of the preceding clauses to the extent possible, which is [−1A, S71C, L166F, S167G, M168L, P171L, S172E, Q173A, G174V, Y179F, A180E, des181] FGF-21.
19. The analogue according to any one of the preceding clauses to the extent possible, which is [−1A, S71C, L166F, S167G, M168L, P171L, S172E, Q173A, G174V, Y179F, A180E] FGF-21.
20. The analogue according to any one of the preceding clauses to the extent possible, which is [−1A, S71C, N121Q, L166F, S167G, M168L, G174V, Y179F, A180E, des181] FGF-21.
21. The analogue according to any one of the preceding clauses to the extent possible, which is [−1A, S71C, N121Q, L166F, S167G, M168L, G174V, Y179F, A180E] FGF-21.
22. The analogue according to any one of the preceding clauses to the extent possible, which is [−1A, S71C, N121Q, L166F, S167G, M168L, P171L, S172E, Q173A, G174V, Y179F, A180E] FGF-21.
23. The analogue according to any one of the preceding clauses to the extent possible, which is [−1A, 121Q, 166F, 167G, 168L, 171L, 172E, 173A, 174V, 179F, 180E, des181] FGF-21.
24. The analogue according to any one of the preceding clauses to the extent possible, which is [−1A, 71C, 121Q, 166F, 167G, 168L, 171L, 172E, 173A, 174V, des179-181] FGF-21.
25. The analogue according to any one of the preceding clauses to the extent possible, which is [−1A, 71C, 121Q, 166F, 168L, 173A, 174V, 179F] FGF-21.
26. The analogue according to any one of the preceding clauses to the extent possible, which is [−1A, S71C, L166F, S167G, M168L, P171L, S172E, Q173A, G174V, Y179F, A180E, des181] FGF-21.
27. The analogue according to any one of the preceding clauses to the extent possible, which is [−1A, 71C, 121Q, 166F, 168L, 174V, 179F, 180E, des181] FGF21.
28. The analogue according to any one of the preceding clauses to the extent possible, which is [−1A, S71C, L166F, S167G, M168L, G174V, Y179F, A180E, des181] FGF-21; [−1A, S71C, L166F, S167G, M168L, G174V, Y179F, A180E] FGF-21; [−1A, S71C, L166F, S167G, M168L, P171L, S172E, Q173A, G174V, Y179F, A180E, des181] FGF-21; [−1A, S71C, L166F, S167G, M168L, P171L, S172E, Q173A, G174V, Y179F, A180E] FGF-21; [−1A, S71C, N121Q, L166F, S167G, M168L, G174V, Y179F, A180E, des181] FGF-21; [−1A, S71C, N121Q, L166F, S167G, M168L, G174V, Y179F, A180E] FGF-21; [−1A, S71C, N121Q, L166F, S167G, M168L, P171L, S172E, Q173A, G174V, Y179F, A180E, des181] FGF-21; [−1A, S71C, N121Q, L166F, S167G, M168L, P171L, S172E, Q173A, G174V, Y179F, A180E] FGF-21; [−1A, 121Q, 166F, 167G, 168L, 171L, 172E, 173A, 174V, 179F, 180E, des181] FGF-21; [−1A, 71C, 121Q, 166F, 167G, 168L, 171L, 172E, 173A, 174V, des179-181] FGF-21, [−1A, 71C, 121Q, 166F, 168L, 173A, 174V, 179F] FGF-21 or [−1A, 71C, 121Q, 166F, 168L, 174V, 179F, 180E, des181] FGF21.
29. The derivative according to any one of the preceding clauses to the extent possible, having a group of the general formula mentioned in clause 1 covalently attached to the sulphur atom in the mercapto group present in the cysteine residue in position 71.
30. The derivative according to any one of the preceding clauses to the extent possible, wherein n is 14.
31. The derivative according to any one of the preceding clauses to the extent possible, wherein n is 16.
32. The derivative according to any one of the preceding clauses to the extent possible, wherein n is 18.
33. The derivative according to any one of the preceding clauses to the extent possible, wherein n is 14, 16 or 18.
34. The derivative according to any one of the preceding clauses to the extent possible, wherein m is 2.
35. The derivative according to any one of the preceding clauses to the extent possible, wherein p is 2.
36. The derivative according to any one of the preceding clauses to the extent possible, wherein q is 2.
37. The derivative according to any one of the preceding clauses to the extent possible, wherein q is 3.
38. The derivative according to any one of the preceding clauses to the extent possible, wherein q is 4.
39. The derivative according to any one of the preceding clauses to the extent possible, wherein n is 14 and m, p and q are each 2.
40. The derivative according to any one of the preceding clauses to the extent possible, wherein n is 16 and m, p and q are each 2.
41. The derivative according to any one of the preceding clauses to the extent possible, wherein n is 18 and m, p and q are each 2.
42. The derivative according to any one of the preceding clauses to the extent possible, in which the parent FGF21 analogue is [−1A, S71C, L166F, S167G, M168L, G174V, Y179F, A180E] FGF21.
43. The derivative according to any one of the preceding clauses to the extent possible, in which the parent FGF21 analogue is [−1A, S71C, L166F, S167G, M168L, G174V, Y179F, A180E, des181] FGF-21; [−1A, S71C, L166F, S167G, M168L, G174V, Y179F, A180E] FGF-21; [−1A, S71C, L166F, S167G, M168L, P171L, S172E, Q173A, G174V, Y179F, A180E, des181] FGF-21; [−1A, S71C, L166F, S167G, M168L, P171L, S172E, Q173A, G174V, Y179F, A180E] FGF-21; [−1A, S71C, N121Q, L166F, S167G, M168L, G174V, Y179F, A180E, des181] FGF-21; [−1A, S71C, N121Q, L166F, S167G, M168L, G174V, Y179F, A180E] FGF-21; [−1A, S71C, N121Q, L166F, S167G, M168L, P171L, S172E, Q173A, G174V, Y179F, A180E, des181] FGF-21; [−1A, S71C, N121Q, L166F, S167G, M168L, P171L, S172E, Q173A, G174V, Y179F, A180E] FGF-21; [−1A, 121Q, 166F, 167G, 168L, 171L, 172E, 173A, 174V, 179F, 180E, des181] FGF-21; [−1A, 71C, 121Q, 166F, 167G, 168L, 171L, 172E, 173A, 174V, des179-181] FGF-21, [−1A, 71C, 121Q, 166F, 168L, 173A, 174V, 179F] FGF-21 or [−1A, 71C, 121Q, 166F, 168L, 174V, 179F, 180E, des181] FGF21.
44. The derivative according to any one of the preceding clauses to the extent possible, in which the parent FGF21 analogue is [−1A, 71C, 121Q, 166F, 167G, 168L, 171L, 172E, 173A, 174V, 179F, 180E, des181] FGF21.
45. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 121Q, 166F, 167G, 168L, 171L, 172E, 173A, 174V, 179F, 180E, des181] Ala-FGF21.
46. The derivative according to any one of the preceding clauses to the extent possible, which is S-71-({2-[2-(2-{2-[2-(2-{2-[(S)-4-carboxy-4-(15-carboxypentadecanoylamino)butyrylamino]ethoxy}ethoxy)-acetylamino]ethoxy}ethoxy)acetylamino]ethylcarbamoyl}methyl) [71C, 121Q, 166F, 167G, 168L, 171L, 172E, 173A, 174V, 179F, 180E, des181] Ala-FGF21.
47. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 121Q, 166F, 167G, 168L, 171L, 172E, 173A, 174V, 179F, 180E] Ala-FGF21.
48. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 121Q, 166F, 167G, 168L, 171L, 172E, 173A, 174V, des179-181] Ala-FGF21.
49. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 121Q, 166F, 167G, 168L, 174V, 179F, 180E, des181] Ala-FGF21.
50. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 121Q, 166F, 168L, 173A, 174V, 179F] Ala-FGF21.
51. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 166F, 167G, 168L, 171L, 172E, 173A, 174V, 179F, 180E, des181] Ala-FGF21.
52. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 166F, 167G, 168L, 174V, 179F, 180E] Ala-FGF21.
53. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 166F, 167G, 168L, 174V, 179F, 180E, des181] Ala-FGF21.
54. The derivative according to any one of the preceding clauses to the extent possible, which is S-71-({2-[2-(2-{2-[2-(2-{2-[(S)-4-carboxy-4-(19-carboxynonadecanoylamino)butyrylamino]ethoxy}ethoxy)-acetylamino]ethoxy}ethoxy)acetylamino]ethylcarbamoyl}methyl) [−1A, 71C, 121Q, 166F, 167G, 168L, 171L, 172E, 173A, 174V, 179F, 180E, des181] Ala-FGF21.
55. The derivative according to any one of the preceding clauses to the extent possible, which is S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [−1A, 71C, 121Q, 166F, 168L, 174V, 179F, 180E, des181] FGF21.
56. The derivative according to any one of the preceding clauses to the extent possible, which is S-71-({2-[2-(2-{2-[2-(2-{2-[(S)-4-carboxy-4-(15-carboxypentadecanoylamino)butyrylamino]ethoxy}ethoxy)-acetylamino]ethoxy}ethoxy)acetylamino]ethylcarbamoyl}methyl) [71C, 121Q, 166F, 167G, 168L, 171L, 172E, 173A, 174V, 179F, 180E, des181] Ala-FGF21; S-71-({2-[2-(2-{2-[2-(2-{2-[(S)-4-carboxy-4-(17-carboxyheptadecanoylamino)butyrylamino]ethoxy}ethoxy)acetylamino]ethoxy}ethoxy)acetylamino]-ethylcarbamoyl}methyl) [71C, 121Q, 166F, 167G, 168L, 171L, 172E, 173A, 174V, 179F, 180E] Ala-FGF21; S-71-({2-[2-(2-{2-[2-(2-{2-[(S)-4-carboxy-4-(17-carboxyheptadecanoylamino)butyrylamino]-ethoxy}ethoxy)acetylamino]ethoxy}ethoxy)acetylamino]ethylcarbamoyl}methyl) [71C, 121Q, 166F, 167G, 168L, 171L, 172E, 173A, 174V, 179F, 180E, des181] Ala-FGF21; S-71-({2-[2-(2-{2-[2-(2-{2-[(S)-4-carboxy-4-(17-carboxyheptadecanoylamino)butyrylamino]ethoxy}ethoxy)acetylamino]ethoxy}-ethoxy)acetylamino]ethylcarbamoyl}methyl) [71C, 121Q, 166F, 167G, 168L, 171L, 172E, 173A, 174V, des179-181] Ala-FGF21; S-71-({2-[2-(2-{2-[2-(2-{2-[(S)-4-carboxy-4-(17-carboxyheptadecanoylamino)-butyrylamino]ethoxy}ethoxy)acetylamino]ethoxy}ethoxy)acetylamino]ethylcarbamoyl}methyl) [71C, 121Q, 166F, 167G, 168L, 174V, 179F, 180E, des181] Ala-FGF21; S-71-({2-[2-(2-{2-[2-(2-{2-[(S)-4-carboxy-4-(17-carboxyheptadecanoylamino)butyrylamino]ethoxy}ethoxy)acetylamino]ethoxy}ethoxy)-acetylamino]ethylcarbamoyl}methyl) [71C, 121Q, 166F, 168L, 173A, 174V, 179F] Ala-FGF21; S-71-({2-[2-(2-{2-[2-(2-{2-[(S)-4-carboxy-4-(17-carboxyheptadecanoylamino)butyrylamino]ethoxy}ethoxy)-acetylamino]ethoxy}ethoxy)acetylamino]ethylcarbamoyl}methyl) [71C, 166F, 167G, 168L, 171L, 172E, 173A, 174V, 179F, 180E, des181] Ala-FGF21; S-71-({2-[2-(2-{2-[2-(2-{2-[(S)-4-carboxy-4-(17-carboxy-heptadecanoylamino)butyrylamino]ethoxy}ethoxy)acetylamino]ethoxy}ethoxy)acetylamino]ethyl-carbamoyl}methyl) [71C, 166F, 167G, 168L, 174V, 179F, 180E] Ala-FGF21; S-71-({2-[2-(2-{2-[2-(2-{2-[(S)-4-carboxy-4-(17-carboxyheptadecanoylamino)butyrylamino]ethoxy}ethoxy)acetylamino]ethoxy}-ethoxy)acetylamino]ethylcarbamoyl}methyl) [71C, 166F, 167G, 168L, 174V, 179F, 180E, des181] Ala-FGF21; S-71-({2-[2-(2-{2-[2-(2-{2-[(S)-4-carboxy-4-(19-carboxynonadecanoylamino)butyrylamino]-ethoxy}ethoxy)acetylamino]ethoxy}ethoxy)acetylamino]ethylcarbamoyl}methyl) [−1A, 71C, 121Q, 166F, 167G, 168L, 171L, 172E, 173A, 174V, 179F, 180E, des181] Ala-FGF21 or S-71-({2-[2-(2-{2-[2-(2-{2-[(S)-4-carboxy-4-(17-carboxyheptadecanoylamino)butyrylamino]ethoxy}ethoxy)acetylamino]ethoxy}-ethoxy)acetylamino]ethylcarbamoyl}methyl) [−1A, 71C, 121Q, 166F, 168L, 174V, 179F, 180E, des181] FGF21.
57. The analogue or derivative according to any one of the preceding clauses to the extent possible which has a potency of at least 1%, preferably at least 5%, more preferably at least 10%, or most preferably at least 20% relative to the potency of Met-FGF21, wherein the potency is determined by measuring glucose uptake in 3T3-L1 adipocytes.
58. The analogue or derivative according to any one of the preceding clauses to the extent possible which has a potency of at least 30%, preferably at least 40%, more preferably at least 50%, even more preferably at least 60%, or most preferably at least 70%, relative to the potency of Met-FGF21.
59. The analogue or derivative according to any one of the preceding clauses to the extent possible which has a potency of at least (i) at least 80%, preferably at least 90%, more preferably at least 100%, even more preferably at least 110%, or most preferably at least 120%, relative to the potency of Met-FGF21.
60. The analogue or derivative according to any one of the preceding clauses to the extent possible which has a potency of at least 100%, preferably at least 120%, more preferably at least 140%, even more preferably at least 160%, or most preferably at least 180%, relative to the potency of Met-FGF21.
61. The analogue or derivative according to any one of the preceding clauses to the extent possible which has a potency of at least 200%, preferably at least 250%, more preferably at least 300%, even more preferably at least 350%, or most preferably at least 400%, relative to the potency of Met-FGF21.
62. A compound according to any one of the preceding clauses, which is any one of the compounds mentioned specifically in the present specification such as in the specific examples, especially any one of examples 1 et seq.
63. A compound according to any one of the preceding clauses for use as a medicament or for use in a medicament.
64. A compound according to any one of the preceding product clauses for treating or preventing diabetes, dyslipidemia, obesity, cardiovascular diseases, metabolic syndrome, and/or Non Alcoholic Fatty Liver Disease (NAFLD).
65. The use of a compound according to any one of the preceding clauses for the preparation of a medicament for the treatment or prevention of diabetes, dyslipidemia, obesity, cardiovascular diseases, metabolic syndrome, and/or Non Alcoholic Fatty Liver Disease (NAFLD).
66. The use of a compound according to any one of the preceding product clauses for the preparation of a pharmaceutical composition for the treatment of diabetes, dyslipidemia, obesity, cardiovascular diseases, metabolic syndrome, and/or Non Alcoholic Fatty Liver Disease (NAFLD).
67. A method of treatment or prevention of diabetes, dyslipidemia, obesity, cardiovascular diseases, metabolic syndrome, and/or Non Alcoholic Fatty Liver Disease (NAFLD), the method comprising administering to a subject in need thereof a therapeutically effective amount of a compound according to any one of the preceding product clauses.
68. Any novel feature or combination of features described herein, especially features described in a clause or in a claim.

Combining one or more of the clauses and embodiments described herein, optionally also with one or more of the claims below, results in further embodiments and the present invention relates to all possible combinations of said clauses, embodiments and claims. In some of the clauses and claims herein, it is mentioned that said claim or clause, respectively, is according to any one of the preceding clauses or claims, respectively, to the extent possible. Any skilled art worker is able to decide to which extent this is possible. Hence, such clauses and claims may only be according to some of the preceding clauses and claims, respectively, even if not specifically stated so. The term “any one of the preceding clauses or claims” covers any logical number of the preceding clauses or claims, respectively, for example one, two, three or four of those preceding clauses and claims, respectively.

The following examples are offered by way of illustration, not by limitation.

### ABBREVIATIONS

The following abbreviations are used in the following, in alphabetical order: BG is blood glucose, BW is body weight, DCM is dichloromethane, DIC is diisopropylcarbodiimide, DIPEA is diisopropylethylamine, DPBS is Dulbecco's Phosphate-Buffered Saline, DVB is divinyl benzene, EDAC is (3-dimethylaminopropyl)ethyl carbodiimide hydrochloride, Fmoc is 9H-fluoren-9-ylmethoxycarbonyl, HEPES is 4-(2-hydroxyethyl)-1-piperazineethanesulfonic acid, HOAt is 1-hydroxy-7-azabenzotriazole, HOBt is 1-hydroxybenzotriazole, HPβCD is hydroxypropyl beta cyclodextrin, HPLC is High Performance Liquid Chromatography, IBMX is 3-isobutyl-1-methylxanthine, Inp is isonipecotic acid, IPTG is isopropyl β-D-1-thiogalactopyranoside check, LCMS is Liquid Chromatography Mass Spectroscopy, MALDI-TOF MS is Matrix-Assisted Laser Desorption/Ionization Time of Flight Mass Spectroscopy, MeOH is methanol, NanoES-MS is Nano-ElectroSpray tandem Mass Spectrometry, NMP is 1-methyl-pyrrolidin-2-one, OEG is 8-amino-3,6-dioxaoctanic acid, OtBu is tert.butyl ester, PBS is phosphate buffered saline, RT is room temperature, TFA is trifluoroacetic acid, TG is triglyceride, THF is tetrahydrofuran, TIPS is triisopropylsilane, Tris is tris(hydroxymethyl)aminomethane or 2-amino-2-hydroxymethylpropane-1,3-diol, Trx is tranexamic acid, TSTU is O—(N-succimidyl)-N,N,N′,N′-tetramethyluronium tetrafluoroborate and HPLC is Ultra Performance Liquid Chromatography.

### General Methods

### LCMS Method 1 (LCMS1)

An Agilent Technologies LC/MSD TOF (G1969A) mass spectrometer was used to identify the mass of the sample after elution from an Agilent 1200 series HPLC system. The deconvolution of the protein spectra was calculated with Agilent's protein confirmation software.

### Eluents:

A: 0.1% Trifluoroacetic acid in water
B: 0.1% Trifluoroacetic acid in acetonitrile

### Column: Zorbax 5u, 300SB-C3, 4.8×50 mm

Gradient: 25%-95% acetonitrile over 15 min

### LCMS Method 2 (LCMS2)

A Perkin Elmer Sciex API 3000 mass spectrometer was used to identify the mass of the sample after elution from a Perkin Elmer Series 200 HPLC system.

### Eluents:

A: 0.05% Trifluoroacetic acid in water
B: 0.05% Trifluoroacetic acid in acetonitrile

### Column: Waters Xterra MS C-18×3 mm id 5 μm

Gradient: 5%-90% acetonitrile over 7.5 min at 1.5 ml/min

### LCMS Method 3 (LCMS3)

A Waters Micromass ZQ mass spectrometer was used to identify the mass of the sample after elution from a Waters Alliance HT HPLC system.

### Eluents:

A: 0.1% Trifluoroacetic acid in water
B: 0.1% Trifluoroacetic acid in acetonitrile

### Column: Phenomenex, Kinetex C18 50×4.60 mm id 2.6 μm, 100AA

Gradient: 10%-90% B over 7.5 min at 1.0 ml/min

### Example 1

### Cloning and Expression of FGF21

The DNA and amino acid sequences for human FGF21 have been disclosed by, e.g., Nishimura et al. in Biochim. Biophys. Acta 1492(1):203-206 (2000). The sequences are also available from public databases with accession nos. EMBL:AB021975 and UNIPROT:Q9NSA1, respectively.

The native polypeptide is synthesised with a signal peptide of 28 amino acids for secretion:

| | |
|---|---|
| 1 | MDSDETGFEH SGLWVSVLAG LLLGACQAHP IPDSSPLLQF GGQVRQRYLY |
| 51 | TDDAQQTEAH LEIREDGTVG GAADQSPESL LQLKALKPGV IQILGVKTSR |
| 101 | FLCQRPDGAL YGSLHFDPEA CSFRELLLED GYNVYQSEAH GLPLHLPGNK |
| 151 | SPHRDPAPRG PARFLPLPGL PPALPEPPGI LAPQPPDVGS SDPLSMVGPS |
| 201 | QGRSPSYAS |

The signal peptide, shown in italics above, is included in the appended sequence listing as SEQ ID NO:2. The mature FGF21 polypeptide consisting of the remaining 181 amino acids is included in the sequence listing as SEQ ID NO:1.

The mature FGF21 polypeptide was cloned and expressed as an intracellular protein in E. coli, without the signal peptide, but with an added N-terminal methionine or an N-terminal Met-Ala which is processed in E. coli resulting in N-terminal Ala (−1Ala). More in particular, a 550 by coding region including at the 3′-end the ATG codon for Met, as well as Nde1 and BamH1 restriction sites at the 3′- and 5′-ends, respectively, was inserted into the expression vector pET 11c in Nde1-BamH1 under control of the phage T7 promoter, and transformed into E. coli B BL21(DE3). The cells were grown in LB amp 100 ug/mL to OD₄₅₀ 0.5, and expression was induced with 0.3 mM IPTG for 4 hours at 37° C. Crude extracts of cells were made by sonication for analysis of FGF21 expression.

A Coomassie stained SDS-PAGE showed successful expression of FGF21 which was identified mainly in the soluble supernatant fraction, with very little in the insoluble pellet. Although the calculated MW of the thus expressed FGF21 (Met-FGF21) (Compound A) is 19.5 kD, it migrated on the gel as a 25 kD protein, which is likely due to the high content of prolines, delaying the movement of the protein.

### Example 2

### Cloning and Expression of FGF21 Analogues

The following, specific analogues of FGF21 can be prepared as is known in the art and expressed in E. coli as generally described in Example 1:

| Analog | |
|---|---|
| Number | Sequence modifications to human FGF21 (Seq. ID 1) |
| 1 | -1A, S71C, L166F, S167G, M168L, G174V, Y179F, A180E, des181 |
| 2 | -1A, S71C, L166F, S167G, M168L, G174V, Y179F, A180E |
| 3 | -1A, S71C, L166F, S167G, M168L, P171L, S172E, Q173A, G174V, Y179F, A180E, |
| | des181 |
| 4 | -1A, S71C, L166F, S167G, M168L, P171L, S172E, Q173A, G174V, Y179F, A180E |
| 5 | -1A, S71C, N121Q, L166F, S167G, M168L, G174V, Y179F, A180E, des181 |
| 6 | -1A, S71C, N121Q, L166F, S167G, M168L, G174V, Y179F, A180E |
| 7 | -1A, S71C, N121Q, L166F, S167G, M168L, P171L, S172E, Q173A, G174V, Y179F, |
| | A180E, des181 |
| 8 | -1A, S71C, N121Q, L166F, S167G, M168L, P171L, S172E, Q173A, G174V, Y179F, |
| | A180E |
| 9 | -1A, 121Q, 166F, 167G, 168L, 171L, 172E, 173A, 174V, 179F, 180E, des181 |
| 10 | -1A, 71C, 121Q, 166F, 167G, 168L, 171L, 172E, 173A, 174V, des179-181 |
| 11 | -1A, 71C, 121Q, 166F, 168L, 173A, 174V, 179F |

The same FGF21 analogues can be expressed and prepared in S. cerevisiae in ways suitable and per se known for this organism.

### Example 3

### Purification of FGF21 Analogues

The FGF21 analogues described in Examples 1-2 may be further purified as follows or using similar techniques:

A slurry (20% w/v) of E. coli in 10 mM potassium phosphate buffer pH 7.5 was sonicated (3 seconds on/off intervals on ice for 5 minutes). The polypeptide was pelleted by centrifugation (10,000×g, for 30 minutes), re-solubilised by sonication in 50 mM Tris pH 8.0, and debris removed by centrifugation (10,000×g, for 30 minutes). The polypeptide in the resulting supernatant was purified by anion exchange chromatography (50 mM Tris pH 8.0, 50-250 mM NaCl) using Q Sepharose Fast Flow resin (GE Healthcare), as generally described in Protein Purification. Principles and Practice Series: Springer Advanced Texts in Chemistry Scopes, Robert K. 3rd ed., 1994. In some instances, further purification was done by size exclusion chromatography using a HiLoad 26/60 Superdex pg 75 column (GE Healthcare) operated with 50 mM Tris pH 8.0 and 200 mM NaCl. For storage the polypeptide was transferred to DPBS, and stored frozen.

Analog number 11: [−1A, S71C, N121Q, L166F, M168L, Q173A, G174V, Y179F] FGF21 LCMS1: Theoretical mass: 19495.03. Found: 19500.40

### Example 4

### Preparation of Reagents which can be Used to Modify the Free Cys of FGF21 Analogues in Position 71

### Preparation of 17-{(S)-1-carboxy-3-[2-(2-{[2-(2-{[2-(2-iodoacetylamino)ethylcarbamoyl]methoxy}-ethoxy)ethylcarbamoyl]methoxy}ethoxy)ethylcarbamoyl]propylcarbamoyl}heptadecanoic acid

Error! Objects Cannot be Created from Editing Field Codes.

### Step 1: 17-[(S)-3-(2-{2-[(2-{2-[(2-Aminoethylcarbamoyl)methoxy]ethoxy}ethylcarbamoyl)methoxy]-ethoxy}ethylcarbamoyl)-1-carboxypropylcarbamoyl]heptadecanoic acid

To a solution of ethanol (10 ml) and ethylenediamine (1 ml) was added 17-((S)-1-carboxy-3-{2-[2-({2-[2-(2,5-dioxopyrrolidin-1-yloxycarbonylmethoxy)ethoxy]ethylcarbamoyl}methoxy)ethoxy]-ethylcarbamoyl}propylcarbamoyl)heptadecanoic acid (500 mg, prepared as described previously in WO2009/083549). After stirring over night at room temperature, the mixture was concentrated in vacuo at 40° C. The residue was purified by preparative HPLC (10-65% acetonitrile, 0.1% TFA, 20 mL/min, C18, 30 mm×250 mm, 110 Å). Yield 332 mg (70%).

LCMS: Theoretical mass: 776.0. Found: 776.6 (M+1).

### Step 2: 17-{(S)-1-Carboxy-3-[2-(2-{[2-(2-{[2-(2-iodoacetylamino)ethylcarbamoyl]methoxy}ethoxy)-ethylcarbamoyl]methoxy}ethoxy)ethylcarbamoyl]propylcarbamoyl}heptadecanoic acid

To a solution of iodoacetic acid (92 mg) in acetonitrile (1 ml) was added TSTU (142 mg) and DIPEA (0.085 ml). After stirring at RT for 60 min a solution of 17-[(S)-3-(2-{2-[(2-{2-[(2-aminoethylcarbamoyl)methoxy]ethoxy}ethylcarbamoyl)methoxy]ethoxy}ethylcarbamoyl)-1-carboxypropylcarbamoyl]heptadecanoic acid (0.320 g) in 0.1M Na2CO3 (12 ml) was added. After stirring for 120 min, pH of the mixture was adjusted to 1 with 1N HCl. The precipitate was filtered off and washed with water and dried in vacuo. Yield 350 mg (90%).

LCMS3: Theoretical mass: 943.9 Found: 944.6 (M+1).

The following reagents can be useful in the modification of FGF analogues, and they can be prepared using similar processes:

| Modifying | |
|---|---|
| Reagent | Structure |
| Number | X = an appropriate leaving group Non-limiting examples are I or Br. |
| I | 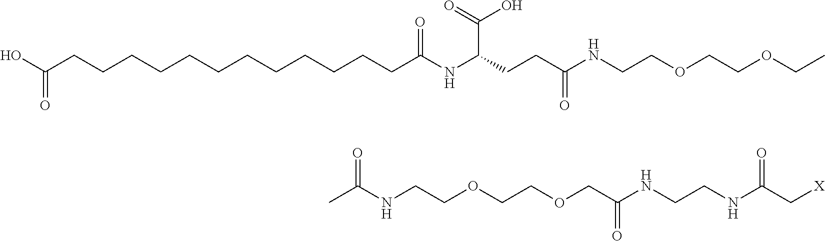 |
| II | 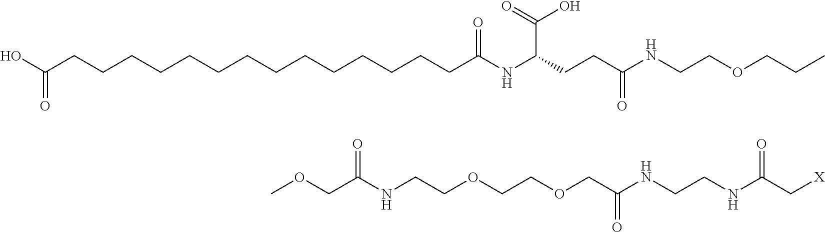 |
| III | 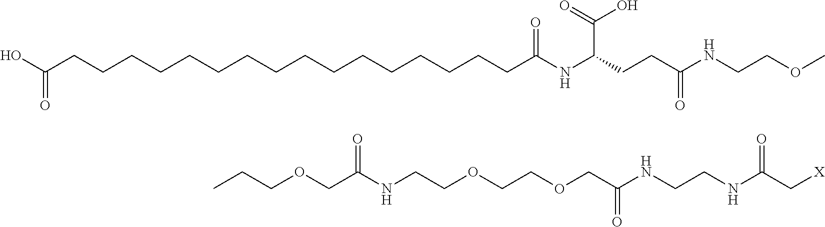 |
| IV | 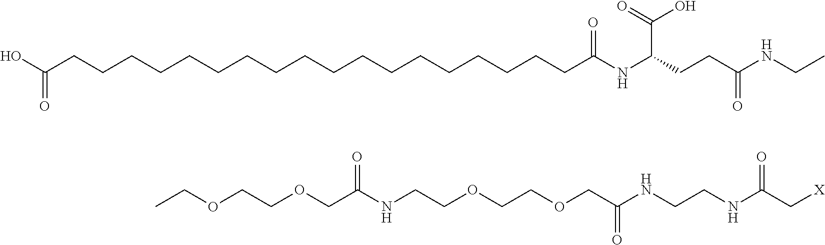 |
| V | 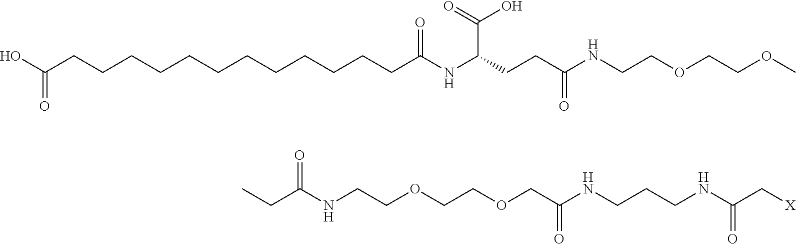 |
| VI | 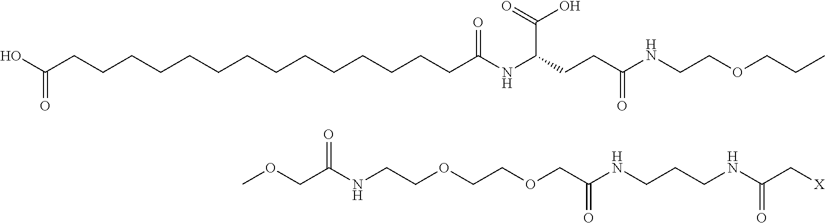 |
| VII | 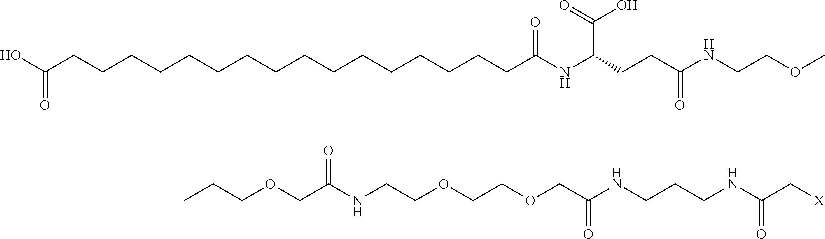 |
| VIII | 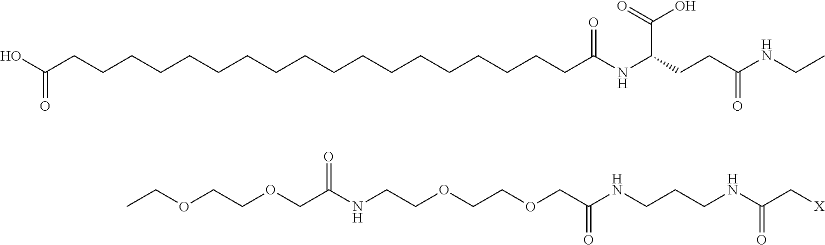 |
| IX | 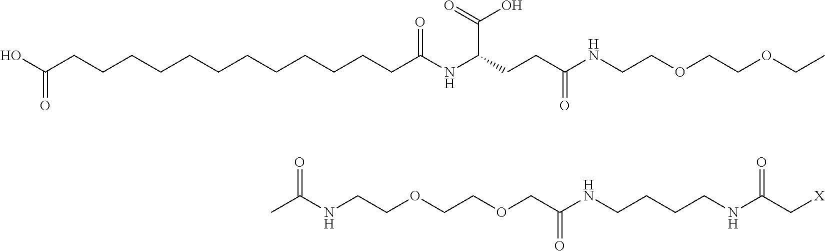 |
| X | 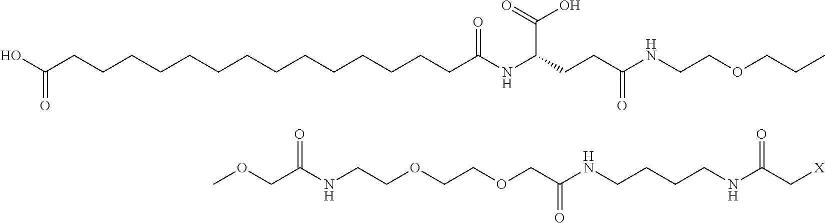 |
| XI | 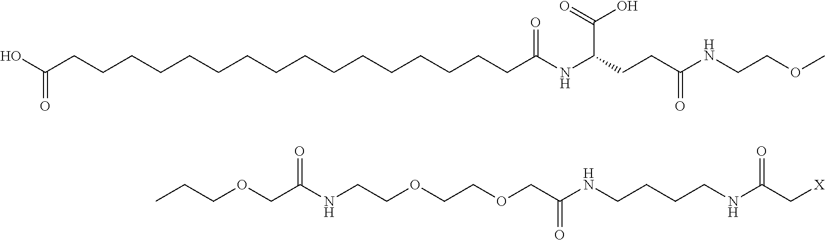 |
| XII | 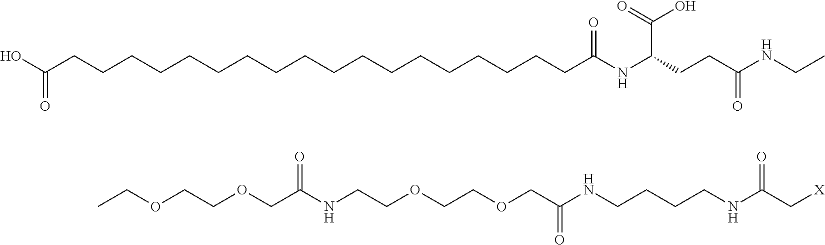 |

### Example 5

### Derivatisation of FGF21 Compounds at 71Cys with Modifying Group

Preparation of a 71Cys derivative of an FGF21 analogue

### Derivative Number 102. Preparation of the (−1A, 71C, 121Q, 166F, 168L, 173A, 174V, 179F) FGF21 derivative S-71-({2-[2-(2-{2-[2-(2-{2-[(S)-4-carboxy-4-(17-carboxyheptadecanoylamino)butyrylamino]-ethoxy}ethoxy)acetylamino]ethoxy}ethoxy)acetylamino]ethylcarbamoyl}methyl) [71C, 121Q, 166F, 168L, 73A, 174V, 179F] Ala-FGF21

The Cys residue at position 71 in the (−1A, 71C, 121Q, 166F, 168L, 173A, 174V, 179F) FGF21 analogue 11, prepared as generally described in Examples 2 and 3, was modified at the thiol group at position 71 with the following reagent prepared as described above:

Error! Objects Cannot be Created from Editing Field Codes.

To [71C, 166F, 167G, 168L, 171L, 172E, 173A, 174V, 179F] Ala-FGF21 (17 mg, 0.00087 mmol), in 20 mM Tris and 0.5M NaCl pH 8.28. 17-{(S)-1-Carboxy-3-[2-(2-{[2-(2-{[2-(2-iodoacetylamino)ethylcarbamoyl]methoxy}ethoxy)ethylcarbamoyl]methoxy}ethoxy)ethylcarbamoyl]propylcarbamoyl}heptadecanoic acid (4.05 mg 0.0035 mmol) in 0.1M NaHCO3 (0.081 ml) was added. After 3 h MiliQ water was added to lower the conductivity to 8.0 mS/cm. The mixture was purified using anion exchange on a MonoQ 10/100 column using A-buffer: 20 mM TRIS, pH 7.5; B-buffer: 20 mM TRIS, 500 mM NaCl, pH 7.5, flow 0.5 mL/min, gradient: 0-100% B over 60CV. The collected fractions were buffer exchanged to a phosphate buffer using a HiPrep 26/10 desalting column. The eluate was collected and filtered through a Millex GV sterile 0.22 um. Yield: 4.65 mg.

MS-TOF: Theoretical mass: 20311.03. Found: 20311.44

In the following table, some specific FGF21 derivatives according to the present invention are illustrated by stating the specific FGF21 analogue and stating the specific modifying agent. In this table, all these compounds of this invention are identified by a derivative number. All these compounds of this invention can be prepared in similar fashion as described above. In this table, any of the specific FGF21 analogues to which the modifying moiety is covalently attached is identified by a “analogue number” which is stated in the table in example 2 above. Furthermore, in this table, any of the reagents used to modify the specific FGF21 analogues is identified by a “modifying reagent number” which is stated in the table in example 4 above. In all the derivatives illustrated in the table below, the modifying reagent reacts with the mercapto group present in Cys in position 71 in the FGF21 analogue.

| Derivative Number | Analog number | Modifying reagent number |
|---|---|---|
| 1. | 1 | I |
| 2. | 2 | I |
| 3. | 3 | I |
| 4. | 4 | I |
| 5. | 5 | I |
| 6. | 6 | I |
| 7. | 7 | I |
| 8. | 8 | I |
| 9. | 1 | II |
| 10. | 2 | II |
| 11. | 3 | II |
| 12. | 4 | II |
| 13. | 5 | II |
| 14. | 6 | II |
| 15. | 7 | II |
| 16. | 8 | II |
| 17. | 1 | III |
| 18. | 2 | III |
| 19. | 3 | III |
| 20. | 4 | III |
| 21. | 5 | III |
| 22. | 6 | III |
| 23. | 7 | III |
| 24. | 8 | III |
| 25. | 1 | IV |
| 26. | 2 | IV |
| 27. | 3 | IV |
| 28. | 4 | IV |
| 29. | 5 | IV |
| 30. | 6 | IV |
| 31. | 7 | IV |
| 32. | 8 | IV |
| 33. | 1 | V |
| 34. | 2 | V |
| 35. | 3 | V |
| 36. | 4 | V |
| 37. | 5 | V |
| 38. | 6 | V |
| 39. | 7 | V |
| 40. | 8 | V |
| 41. | 1 | VI |
| 42. | 2 | VI |
| 43. | 3 | VI |
| 44. | 4 | VI |
| 45. | 5 | VI |
| 46. | 6 | VI |
| 47. | 7 | VI |
| 48. | 8 | VI |
| 49. | 1 | VII |
| 50. | 2 | VII |
| 51. | 3 | VII |
| 52. | 4 | VII |
| 53. | 5 | VII |
| 54. | 6 | VII |
| 55. | 7 | VII |
| 56. | 8 | VII |
| 57. | 1 | VIII |
| 58. | 2 | VIII |
| 59. | 3 | VIII |
| 60. | 4 | VIII |
| 61. | 5 | VIII |
| 62. | 6 | VIII |
| 63. | 7 | VIII |
| 64. | 8 | VIII |
| 65. | 1 | IX |
| 66. | 2 | IX |
| 67. | 3 | IX |
| 68. | 4 | IX |
| 69. | 5 | IX |
| 70. | 6 | IX |
| 71. | 7 | IX |
| 72. | 8 | IX |
| 73. | 1 | X |
| 74. | 2 | X |
| 75. | 3 | X |
| 76. | 4 | X |
| 77. | 5 | X |
| 78. | 6 | X |
| 79. | 7 | X |
| 80. | 8 | X |
| 81. | 1 | XI |
| 82. | 2 | XI |
| 83. | 3 | XI |
| 84. | 4 | XI |
| 85. | 5 | XI |
| 86. | 6 | XI |
| 87. | 7 | XI |
| 88. | 8 | XI |
| 89. | 1 | XII |
| 90. | 2 | XII |
| 91. | 3 | XII |
| 92. | 4 | XII |
| 93. | 5 | XII |
| 94. | 6 | XII |
| 95. | 7 | XII |
| 96. | 8 | XII |
| 97. | 10 | I |
| 98. | 11 | I |
| 99. | 10 | II |
| 100. | 11 | II |
| 101. | 10 | III |
| 102. | 11 | III |
| 103. | 10 | IV |
| 104. | 11 | IV |
| 105. | 10 | V |
| 106. | 11 | V |
| 107. | 10 | VI |
| 108. | 11 | VI |
| 109. | 10 | VII |
| 110. | 11 | VII |
| 111. | 10 | VIII |
| 112. | 11 | VIII |
| 113. | 10 | IX |
| 114. | 11 | IX |
| 115. | 10 | X |
| 116. | 11 | X |
| 117. | 10 | XI |
| 118. | 11 | XI |
| 119. | 10 | XII |
| 120. | 11 | XII |

For example, Derivative 102 could be prepared by reacting analogue 11 which is (−1A, S71C, 121Q, L166F, M168L, Q173A, G174V, Y179F)-FGF21 together with 17-{(S)-1-carboxy-3-[2-(2-{[2-(2-{[4-(2-iodoacetylamino)butylcarbamoyl]methoxy}ethoxy)ethylcarbamoyl]methoxy}ethoxy)ethylcarbamoyl]-propylcarbamoyl}heptadecanoic acid, such that the free thiol of the cysteine at position 71 becomes modified with the modifying moiety III ({4-[2-(2-{2-[2-(2-{2-[(S)-4-carboxy-4-(17-carboxyheptadecanoylamino)butyrylamino]ethoxy}ethoxy)acetylamino]ethoxy}ethoxy)acetylamino]butylcarbamoyl}methyl) thus forming S-71-{4-[2-(2-{2-[2-(2-{2-[(S)-4-carboxy-4-(17-carboxyheptadecanoylamino)butyrylamino]-ethoxy}ethoxy)acetylamino]ethoxy}ethoxy)acetylamino]butylcarbamoyl}methyl [S71C, 121Q, L166F, M168L, G174V, Y179F] Ala-FGF21.

In the compounds of this invention with the above derivative numbers 1-120, the modifying moiety covalently attached to the sulphur atom from the mercapto group in 71Cys is as stated in the following table using the same “modifying moiety numbers” as used for the corresponding modifying reagents.

| Modifying | |
|---|---|
| moiety | |
| Number | Modifying moiety |
| I | {2-[2-(2-{2-[2-(2-{2-[(S)-4-Carboxy-4-(13-carboxytridecanoylamino)butyrylamino]ethoxy}- |
| | ethoxy)acetylamino]ethoxy}ethoxy)acetylamino]ethylcarbamoyl}methyl |
| II | {2-[2-(2-{2-[2-(2-{2-[(S)-4-Carboxy-4-(15-carboxypentadecanoylamino)butyrylamino]- |
| | ethoxy}ethoxy)acetylamino]ethoxy}ethoxy)acetylamino]ethylcarbamoyl}methyl |
| III | {2-[2-(2-{2-[2-(2-{2-[(S)-4-Carboxy-4-(17-carboxyheptadecanoylamino)butyrylamino]- |
| | ethoxy}ethoxy)acetylamino]ethoxy}ethoxy)acetylamino]ethylcarbamoyl}methyl |
| IV | {2-[2-(2-{2-[2-(2-{2-[(S)-4-Carboxy-4-(19-carboxynonadecanoylamino)butyrylamino]- |
| | ethoxy}ethoxy)acetylamino]ethoxy}ethoxy)acetylamino]ethylcarbamoyl}methyl |
| V | {3-[2-(2-{2-[2-(2-{2-[(S)-4-Carboxy-4-(13-carboxytridecanoylamino)butyrylamino]ethoxy}- |
| | ethoxy)acetylamino]ethoxy}ethoxy)acetylamino]propylcarbamoyl}methyl |
| VI | {3-[2-(2-{2-[2-(2-{2-[(S)-4-Carboxy-4-(15-carboxypentadecanoylamino)butyrylamino]- |
| | ethoxy}ethoxy)acetylamino]ethoxy}ethoxy)acetylamino]propylcarbamoyl}methyl |
| VII | {3-[2-(2-{2-[2-(2-{2-[(S)-4-Carboxy-4-(17-carboxyheptadecanoylamino)butyrylamino]- |
| | ethoxy}ethoxy)acetylamino]ethoxy}ethoxy)acetylamino]propylcarbamoyl}methyl |
| VIII | {3-[2-(2-{2-[2-(2-{2-[(S)-4-Carboxy-4-(19-carboxynonadecanoylamino)butyrylamino]- |
| | ethoxy}ethoxy)acetylamino]ethoxy}ethoxy)acetylamino]propylcarbamoyl}methyl |
| IX | {4-[2-(2-{2-[2-(2-{2-[(S)-4-Carboxy-4-(13-carboxytridecanoylamino)butyrylamino]ethoxy}- |
| | ethoxy)acetylamino]ethoxy}ethoxy)acetylamino]butylcarbamoyl}methyl |
| X | {4-[2-(2-{2-[2-(2-{2-[(S)-4-Carboxy-4-(15-carboxypentadecanoylamino)butyrylamino]- |
| | ethoxy}ethoxy)acetylamino]ethoxy}ethoxy)acetylamino]butylcarbamoyl}methyl |
| XI | {4-[2-(2-{2-[2-(2-{2-[(S)-4-Carboxy-4-(17-carboxyheptadecanoylamino)butyrylamino]- |
| | ethoxy}ethoxy)acetylamino]ethoxy}ethoxy)acetylamino]butylcarbamoyl}methyl |
| XII | {4-[2-(2-{2-[2-(2-{2-[(S)-4-Carboxy-4-(19-carboxynonadecanoylamino)butyrylamino]- |
| | ethoxy}ethoxy)acetylamino]ethoxy}ethoxy)acetylamino]butylcarbamoyl}methyl |

### Example 6

### Potency Assay—Glucose Uptake in 3T3-L1 Adipocytes

The following assay was used for determining the biological activity, or potency, of FGF21 compounds of the invention.

Mouse 3T3-L1 fibroblasts (e.g. available from ATCC, catalogue no. CL-173) are maintained in basal medium (DMEM (4500 mg/l Glucose) with 10% Fetal Bovine Serum (FBS) and Penicillin/Streptomycin). The cells are not allowed to reach confluence and should be passed (transferred to new vials) before reaching approx. 60% of confluency (by visual inspection).

For the glucose uptake assay, cells are plated 80,000 cells/well in a 24 well plate, or 20,000 cells/well in a 96 well plate, and when they reach confluency (high density, with a view to have differentiated adipose cells made), the medium is changed from basal medium to basal medium containing Troglitazone, IBMX, Dexamethasone (commercially available from, e.g., Sigma) and human insulin (commercially available from, e.g., Novo Nordisk A/S).

The cells are used 7-14, preferably 7-10, days after initiation of differentiation. The cells are stimulated with increasing concentrations (0-300 nM) of the FGF21 polypeptides or derivatives of the invention for 20 hours in basal medium. Before addition of 3H-deoxyglucose (in what follows: the tracer) the cells are washed in warm (approximately 37° C.) assay buffer (PBS with 1 mM MgCl₂ and 2 mM CaCl₂), HEPES and 0.1% Human serum albumin) and the cells are incubated with the tracer for 1 hour. This incubation is terminated by washing twice in ice cold assay buffer. The cells are lysed with Triton X-100 and lysates transferred to a 96 wells plate, microscint-40 (commercially available from, e.g., Perkin Elmer) is added and amount of tracer counted in a TOP-counter (e.g. a Packard top-counter from Perkin Elmer). The EC₅₀ of the polypeptide in question is calculated. The results which are shown in Table 1 below indicate the EC₅₀ (potency) of the FGF21 compounds of the invention relative to that of Met-FGF21.

TABLE 1
Potency of FGF21 compounds

| | Glucose uptake 3T3-L1 |
|---|---|
| Analog or derivative ID | Potency (%) rel. to Met-FGF21 |
| Met-FGF21 | 100 |
| Analog 9 | 1594 |
| Analog 8 | 1099 |
| Derivative 24 | 796 |
| Analog 7 | 734 |
| Derivative 23 | 445 |
| Derivative 31 | 995 |
| Analog 10 | 121 |
| Derivative 101 | 13 |
| Analog 6 | 1017 |
| Analog 5 | 571 |
| Derivative 21 | 270 |
| Analog 11 | 529 |
| Derivative 102 | 484 |
| Analog 4 | 102 |
| Analog 3 | 383 |
| Derivative 19 | 57 |
| Analog 2 | 1325 |
| Derivative 18 | 381 |
| Analog 1 | 1190 |
| Derivative 17 | 438 |

It appears from the results of Table 1 that, generally, the FGF21 compounds of the invention have an improved potency as compared to the potency of Met-FGF21.

### Example 7

### HEK293/Beta-Klotho Erk Phosphorylation Assay

Erk phosphorylation assay was performed in HEK293 cells that were stably transfected with human beta-Klotho. The HEK293T/b-klotho stable cells were seeded at 30000 cells/well on 96-well plates. After two days, fresh media was added, and after 2 hours more the FGF21 proteins were added. The plates were incubated for 12 minutes. And total ERK phosphorylation was assessed using an AlphaScreen SureFire Phospho-ERK1/2 Assay Kit (Perkin Elmer, Waltham, Mass.) according to the manufacturer's instructions and an EnVision Multilabel Microplate Reader Model 2103 (Perkin Elmer) with the AlphaScreen HTS Turbo option was used for signal detection. Data are represented as means+/−S.E.M. EC50 values were determined from a 4-parameter logistic nonlinear regression analysis using GraphPad Prism version 5.02. References: Yie, J. et al.: FGF21 N- and C-termini play different roles in receptor interaction and activation, FEBS Letters 583 (2009) 19-24, and Micanovic R. et al.: Different roles of N- and C-termini in the functional activity of FGF21. J. Cell. Physiol. 2009 May; 219(2):227-34.

TABLE 2
ERK

| | pERK-HEK293-Beta-klotho |
|---|---|
| | without HSA [EC50 (nM)] |
| Analog or derivative ID | Median Value |
| Met-FGF21 | 1.6 |
| Analog 9 | 0.70 |
| Analog 8 | 0.76 |
| Derivative 24 | 1.01 |
| Analog 7 | 0.63 |
| Derivative 23 | 0.64 |
| Derivative 31 | 1.46 |
| Analog 10 | 12.51 |
| Derivative 101 | 23.8 |
| Analog 6 | 0.71 |
| Analog 5 | 0.78 |
| Derivative 21 | 1.07 |
| Analog 11 | 0.98 |
| Derivative 102 | 1.40 |
| Analog 4 | 0.47 |
| Analog 3 | 0.77 |
| Derivative 19 | 0.48 |
| Analog 2 | 0.64 |
| Derivative 18 | 0.47 |
| Analog 1 | 0.71 |
| Derivative 17 | 0.98 |

For [−1A, 71C, 121Q, 166F, 168L, 174V, 179F, 180E, des181] FGF21, the Erk value is 0.97. This analogue can, for example, be derivatised by covalently attaching the above modifying moiety number III being {2-[2-(2-{2-[2-(2-{2-[(S)-4-carboxy-4-(17-carboxyheptadecanoylamino)butyrylamino]ethoxy}-ethoxy)acetylamino]ethoxy}ethoxy)acetylamino]ethylcarbamoyl}methyl to the sulphur atom from the mercapto group in 71Cys.

### Example 8

### In vivo test of FGF21 compounds—pharmacodynamics

The db/db mouse is a mouse model for Type 2 diabetes. The mice lack the leptin receptor and they are characterized by hyperglycemia, insulin resistance, hyperphagia and obesity.

Male db/db mice (9-10 weeks old) were used to measure the effect on blood glucose and body weight of the following FGF21 analogue and derivatives.

The compounds were administered s.c. 0.1 mg/kg in 50 mM phosphate, 145 mM NaCl, 0.05% Tween-80, pH=7.4 (2 ml/kg) once daily for 7 days (n=7-9). The respective vehicle treated groups (control) were treated with 50 mM phosphate, 145 mM NaCl, 0.05% Tween-80, pH=7.4, (2 ml/kg) s.c. once daily for 7 days (n=8-9). Body weight was measured before dosing and again after 7 days treatment. Non-fasting blood glucose was measured before dosing and again 2 hours after dosing day 7. Blood glucose was measured using a glucose analyzer (Biosen 5040) based on the glucose oxidase method. The results are shown in Table 1 below.

TABLE 3
Difference from vehicle in delta blood
glucose and delta body weight (day 1-7)

| | Δ blood glucose | Δ body weight |
|---|---|---|
| Analogue 7 # | −9.72 ± 0.66 *** | −0.38 ± 0.26 |
| Derivative 23 | −11.38 ± 0.79 *** | −2.03 ± 0.19 *** |
| Derivative 101 | −10.31 ± 0.60 *** | −1.07 ± 0.12 ** |
| Derivative 24 | −12.21 ± 0.95 *** | −1.60 ± 0.20 *** |
| Derivative 17 | −10.61 ± 0.58 *** | −1.74 ± 0.35 *** |
| Derivative 19 | −12.47 ± 0.92 *** | −1.90 ± 0.59 ** |
| Derivative 31 | −12.80 ± 0.92 *** | −1.37 ± 0.28 ** |
| Derivative 18 | −10.87 ± 1.03 *** | −0.63 ± 0.25 |
| Derivative 102 | −12.63 ± 0.62 *** | −1.11 ± 0.23 * |

# dosing BID
* p < 0.05,
** p < 0.01,
*** p < 0.001 Student's t-test comparing delta value of compound vs. respective vehicle, n = 7-9

The results in table 3 show that the FGF21 derivatives of the invention are biologically active in vivo, with effective lowering of body weight and non-fasting/fasting blood glucose.

### Example 9

### In Vivo Test for FGF21 Compounds—Pharmacokinetics

### Mini Pig

The pharmacokinetic profile of Met-FGF21 can be tested in normal male Gottingen mini pigs, n=4 (12-15 months old, 25 kg). The plasma concentration of the compound to be tested is monitored for 14 days. The Met-FGF21 is dosed as a single intravenous dose of 0.1 mg/kg (approximately 5 nmol/kg).

The mean half-life (T_{y}) of the comparative compound Met-FGF21 has been determined to be 10.8 hours with a standard deviation of 2.7 hours.

The pharmokinetic profile of the FGF21 compound of the invention is tested in normal male Gottingen mini pigs, n=4 (12-15 months old, 25 kg). The plasma concentration is monitored for 19 days. The compound is dosed as a single intravenous dose of 0.05 mg/kg (approximately 2.5 nmol/kg).

The mean half-life (T_{1/2}) of the compound to be tested is determined.

The plasma levels of the FGF21 compounds can be determined using Fibroblast Growth Factor-21 Human ELISA (available from BioVendor, catalogue no. RD191108200R). The PC based software, WinNonLin version 5.2 from Pharsight Corportion, Cary N.C., can be used for the pharmacokinetic calculation.

This test will confirm the protracted effect of the FGF21 derivatives of this invention.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

The citation and incorporation of patent documents herein is done for convenience only and does not reflect any view of the validity, patentability, and/or enforceability of such patent documents. The mentioning herein of references is no admission that they constitute prior art.

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Herein, the word “comprise” is to be interpreted broadly meaning “include”, “contain” or “comprehend” (vide, EPO guidelines C, III, 4.13).

This invention includes all modifications and equivalents of the subject matter recited in the claims and clauses appended hereto as permitted by applicable law.

What is claimed is:

1. [−1A, L166F, M168L, G174V, Y179F] FGF21, analogues of [−1A, L166F, M168L, G174V, Y179F] FGF21 optionally containing one or more of the following amino acid substitutions (exchanges): 71C, 121Q, 173A and/or des181, optionally, having up to four further mutations and/or, optionally, the 179 and/or 180 amino acid is not present and derivatives of such analogues containing Cys in position 71 which derivatives have a group of the general formula HOOC—(CH₂), —CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71.

2. [−1A, 71C, L166F, M168L, G174V, Y179F] FGF21, analogues of [−1A, 71C, L166F, M168L, G174V, Y179F] FGF21 optionally containing one or more of the following amino acid substitutions (exchanges): 121Q, 173A and/or des181, optionally, having up to four further mutations and/or, optionally, the 179 and/or 180 amino acid is not present and derivatives of such analogues which derivatives have a group of the general formula HOOC—(CH₂)ₙ—CONH—CH(COOH)—CH₂—CH₂—CONH—(CH₂CH₂O)ₘ—CH₂—CONH—(CH₂CH₂O)ₚ—CH₂—CONH—(CH₂)_{q}—NHCO—CH₂— (modifying moiety), wherein n is an integer in the range 10-20, m is an integer in the range 1-3, p is an integer in the range 1-3, and q is an integer in the range 2-4, covalently attached to the sulphur atom in the mercapto group present in the cysteine residue in position 71, according to the previous claim.

3. The derivative according to claim 1, wherein n is 14.

4. The derivative according to claim 1, wherein n is 16.

5. The derivative according to claim 1, wherein n is 18.

6. The derivative according to claim 1, wherein q is 2

7. The derivative according to claim 1, wherein q is 3.

8. The derivative according to claim 1, wherein q is 3.

9. The derivative according to claim 1, wherein q is 4.

10. The derivative according to claim 2, wherein n is 14.

11. The derivative according to claim 2, wherein n is 16.

12. The derivative according to claim 2, wherein n is 18.

13. The derivative according to claim 2, wherein q is 2

14. The derivative according to claim 2, wherein q is 3.

15. The derivative according to claim 2, wherein q is 3.

16. The derivative according to claim 2, wherein q is 4.

17. A method of treating diabetes, dyslipidemia, obesity, cardiovascular diseases, metabolic syndrome, and/or Non Alcoholic Fatty Liver Disease (NAFLD) comprising administering an effective amount of the derivative of claim 1 to a subject in need thereof.

18. A method of treating diabetes, dyslipidemia, obesity, cardiovascular diseases, metabolic syndrome, and/or Non Alcoholic Fatty Liver Disease (NAFLD) comprising administering an effective amount of the derivative of claim 2 to a subject in need thereof.


SEQUENCE LISTING (2 sequences)

SEQ ID NO: 1 - protein, 181 aa, Homo sapiens
  mat_peptide (1)..(181)
  DISULFID (75)..(93)
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 28 aa, Homo sapiens
  SIGNAL (1)..(28)
MDSDETGFEHSGLWVSVLAGLLLGACQA